(19) United States
(12) Patent Application Publication
(10) Pub. No.: US 2010/0290952 A1
(43) Pub. Date: Nov. 18, 2010

(54) SAMPLE ANALYZER AND BLOOD COAGULATION ANALYZER

(75) Inventors: Hiroki Koike, Kobe-shi (JP); Norimasa Yamamoto, Kobe-shi (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION, Kobe-shi (JP)

(21) Appl. No.: 12/775,150

(22) Filed: May 6, 2010

(30) Foreign Application Priority Data
May 12, 2009 (JP) 2009-115663

(51) Int. Cl. G01N 33/50; G01N 21/59

(52) U.S. Cl. 422/73; 356/436

(57) ABSTRACT

The present invention is to present a sample analyzer, including: a lamp member which includes a halogen lamp including an electrode and a filament connected to the electrode; a holding mechanism for holding the lamp member such that the filament of the halogen lamp is positioned above the electrode; a light receiver for receiving light irradiated from the halogen lamp through an analysis sample; and an analysis unit for analyzing a component contained in the analysis sample based on the light received by the light receiver.

### FIELD OF THE INVENTION

The present invention relates to a sample analyzer with a halogen lamp, and a blood coagulation analyzer

### BACKGROUND

There are conventionally known sample analyzers provided with halogen lamps. It is generally known that in these sample analyzers, after reagent has been added to a specimen to prepare an analysis sample, the analysis sample is irradiated by light of a particular wavelength, and the scattered light and transmitted light from the analysis sample is analyzed to obtain analysis data.

United States Patent Publication No. 2008/158552 discloses an analyzer provided with a halogen lamp with a filament and electrode, lamp housing to accommodate the halogen lamp and which has an illumination port for directing the light irradiated from the halogen lamp to the outside, and a control unit for analyzing a component contained in the analysis sample based on the light irradiated from the halogen lamp through the irradiation port provided in the lamp housing. In this analyzer, a lamp insertion hole is provided on the top surface of the lamp housing to allow insertion of the halogen lamp from above into the interior of the lamp housing. The halogen lamp is accommodated in the lamp housing with the filament positioned below the electrode via the insertion of the halogen lamp through the lamp insertion hole into the interior of the lamp housing.

However, the analyzer disclosed in United States Patent Publication No. 2008/158552 requires frequent replacement of the halogen lamp due to the short service life of the lamp. Since sample measurements can not be performed while the halogen lamp is being replaced, there is demand for a halogen lamp that has a longer service life so as to reduce the frequency of lamp replacement.

### SUMMARY

The scope of the present invention is defined solely by the appended claims, and is not affected to any degree by the statements within this summary.

A first aspect of the present invention is a sample analyzer, comprising: a lamp member comprising a halogen lamp including an electrode and a filament connected to the electrode; a holding mechanism for holding the lamp member such that the filament of the halogen lamp is positioned above the electrode; a light receiver for receiving light irradiated from the halogen lamp through an analysis sample; and an analysis unit for analyzing a component contained in the analysis sample based on the light received by the light receiver.

A second aspect of the present invention is a blood coagulation analyzer, comprising: a sample preparing unit for preparing an analysis sample from a blood sample and a reagent for blood coagulation analysis; a light source for irradiating light on the analysis sample prepared by the sample preparing unit; a light receiver for receiving light irradiated from the light source through the analysis sample; an analysis unit for analyzing a component contained in the analysis sample based on the light received by the light receiver, the component being related to coagulation function of the blood sample, wherein the light source comprises: a lamp member comprising a halogen lamp including an electrode and a filament connected to the electrode; and a holding mechanism for holding the lamp member such that the filament of the halogen lamp is positioned above the electrode.

### BRIEF DESCRIPTION OF THE DRAWINGS

FIG. 1 is a perspective view showing the general structure of an embodiment of the sample analyzer of the present invention;

FIG. 2 is a top view showing the detection device and transport device of the sample analyzer of the embodiment in FIG. 1;

FIG. 3 is a block diagram showing the structure of the optical information obtainer of the sample analyzer of the embodiment in FIG. 1;

FIG. 4 is an exploded perspective view showing the structure of the lamp unit of the optical information obtainer of the embodiment of the sample analyzer in FIG. 1;

FIG. 5 is a perspective view showing the structure of the lamp unit of the optical information obtainer of the embodiment of the sample analyzer in FIG. 1;

FIG. 6 is a perspective view illustrating the structure of the base of the lamp unit of the optical information obtainer of the embodiment of the sample analyzer in FIG. 1;

FIG. 7 is a brief view showing the structure of the lamp unit of the optical information obtainer of the embodiment of the sample analyzer in FIG. 1;

FIG. 8 is a perspective view showing the structure of the halogen lamp of the lamp unit of the embodiment of the sample analyzer in FIG. 1;

FIG. 9 is a cross sectional view showing the structure of the vicinity of the halogen lamp and the lamp housing of the lamp unit of the embodiment of the sample analyzer in FIG. 1;

FIG. 10 is a cross sectional view showing the structure of the vicinity of the halogen lamp and the lamp housing of the lamp unit of the embodiment of the sample analyzer in FIG. 1;

FIG. 11 is a bottom view showing the structure of the vicinity of the halogen lamp and the lamp housing of the lamp unit of the embodiment of the sample analyzer in FIG. 1;

FIG. 12 is a frontal view showing the structure of the vicinity of the halogen lamp and the lamp housing of the lamp unit of the embodiment of the sample analyzer in FIG. 1;

FIG. 13 is a rear view showing the structure of the vicinity of the halogen lamp and the lamp housing of the lamp unit of the embodiment of the sample analyzer in FIG. 1;

FIG. 14 is a side view showing the structure of the vicinity of the halogen lamp and the lamp housing of the lamp unit of the embodiment of the sample analyzer in FIG. 1;

FIG. 15 is a schematic view illustrating the halogen cycle of the halogen lamp of the lamp unit of the embodiment of the sample analyzer in FIG. 1;

FIG. 16 illustrates the effect based on service life tests of the halogen lamp of the lamp unit of the embodiment of the sample analyzer in FIG. 1; and

FIG. 17 is a perspective view showing the structure of the base of the lamp unit of the optical information obtainer of a modification of the embodiment of the sample analyzer in FIG. 1.

### DETAILED DESCRIPTION OF THE EMBODIMENT

The embodiment of the present invention is described hereinafter based on the drawings.

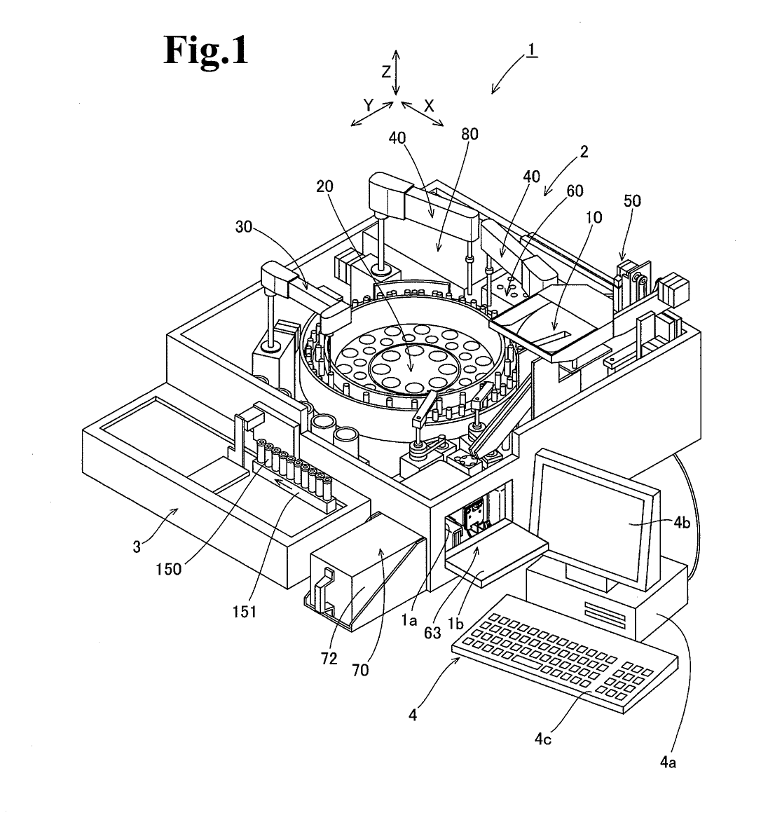

The general structure of the embodiment of the sample analyzer 1 of the present invention is described below with reference to FIGS. 1 through 14.

The embodiment of the sample analyzer 1 of the present invention is an analyzer for optically measuring and analyzing the amount and degree of activity of a specific substance related to blood coagulation and fibrinolytic function, and uses blood plasma as the sample. Note that in the sample analyzer 1 of the present embodiment, optical measurement of a sample is performed using coagulation time, synthetic substrate, and immunoturbidity methods. The coagulation time method is a measurement method that detects the process of the sample coagulation as a change in transmitted light or scattered light. In the coagulation time method, light irradiates an analysis sample prepared from blood plasma and reagent, and the change in turbidity that occurs when the fibrinogen in the analysis sample is transformed to fibrin is detected as a change in the transmitted light. The synthetic substrate method is a measurement method for detecting the change in absorbed light in the process of coloring by a synthetic substrate colorant added to a sample based on the change in light transmission. The immunoturbidity method is a measurement method for detecting the change in absorbed light via an antibody-antigen reaction of an antibody sensitive reagent such as latex added to the sample based on the change in light transmission. Measurement criteria of the coagulation time method include, PT (prothrombin time), PTT (partial thromboplastin time), APTT (activated partial thromboplastin time), and Fbg (fibrinogen quantity), LA (lupus anticoagulant) and the like. Furthermore, measurement items of the synthetic substrate method include ATIII and the like, and measurement items of the immunoturbidity method include D dimer, FDP and the like.

The sample analyzer 1 is configured by a detection device 2, transport device 3 that is disposed on the front side of the detection device 2, and a control device 4 that is electrically connected to the detection device 2, as shown in FIG. 1.

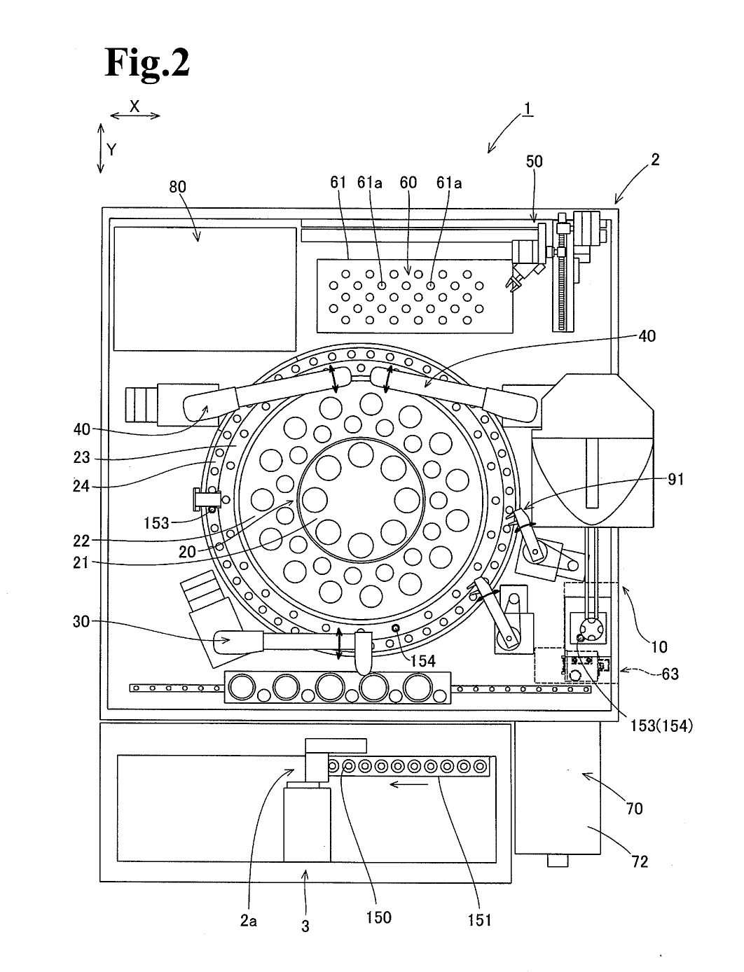

The transport device 3 automatically supplies a sample to the detection device 2 by transporting a rack 151 holding a plurality (10 in the present embodiment) of test tubes 150 containing samples to a position corresponding to the aspiration/dispensing position 2a (refer to FIG. 2) of the detection device 2.

The control device 4 is a personal computer (PC) that includes a controller 4a, display unit 4b, and keyboard 4c, as shown in FIG. 1. The controller 4a controls the operations of the detection device 2 and transport device 3, and has the function of analyzing the optical information of the sample obtained by the detection device 2. Specifically, the controller 4a of the control device 4, has the function of analyzing a component contained in the analysis sample based on the light received by a photoelectric conversion element 64 which is described later. For example, the controller 4a monitors the amount of transmission light received by the photoelectric conversion element 64, and calculates the concentration of fibrinogen contained in the analysis sample based on the change in the amount of transmission light during a predetermined time period. The controller 4a is configured by a CPU, ROM, RAM and the like. The display unit 4b is provided to display the analysis result obtained by the controller 4a.

The detection device 2 is capable of obtaining optical information related to a sample by optically measuring the sample supplied from the transport device 3. In the sample analyzer 1 of the present embodiment, optical measurement is performed on a sample dispensed into cuvettes 153 and 154 (refer to FIG. 2) of the detection device 2 from the test tube 150 of the transport device 3. The cuvette 153 is held on a primary dispensing table 24 to be described later, and the cuvette 154 is held in a secondary dispensing table 23 to be described later. The detection device 2 is provided with a cuvette supplier 10, transporter 20, sample dispensing arm 30, two reagent dispensing arms 40, cuvette mover 50, optical information obtainer 60, and cuvette discard unit 70, as shown in FIGS. 1 and 2.

The cuvette supplier 10 is capable of sequentially supplying a plurality of cuvettes 153 and 154 to the transporter 20, as shown in FIG. 2.

The transporter 20 is provided to transport, in a rotational direction, the test tube (not shown in the drawing) containing the reagent to be added tot he sample in the cuvettes 153 and 154 supplied from the cuvette supplier 10. The transporter 20 is configured by a circular reagent table 21, annular reagent table 22 disposed on the outer side of the circular reagent table 21, annular secondary dispensing table 23 disposed on the outer side of the annular reagent table 22, and annular primary dispensing table 24 disposed on the outer side of the annular secondary dispensing table 23.

The reagent tables 21 and 22 are respectively capable of holding a plurality of test tubes (not shown) containing various reagents to be added when preparing an analysis sample from a specimen. Reagents to be used in the measurement of measurement items PT, APTT, Fbg and the like are held in the reagent tables 21 and 22. The primary dispensing table 24 and the secondary dispensing table 23 are respectively capable of holding the cuvettes 153 and 153 supplied from the cuvette supplier 10. A specimen is dispensed from the test tube 150 of the transporting device 3 to the cuvette 153 held in the primary dispensing table 24 when performing the primary dispensing process. A specimen is dispensed from the cuvette 153 held on the primary dispensing table 24 into the cuvette 154 held in the secondary dispensing table 23 when performing the secondary dispensing process.

The sample dispensing arm 30 has the function of dispensing the sample in the test tube 150, which has been transported by the transport device 3 to the aspiration/dispensing position 2a of the detection device 2, into the cuvette 153 held in the primary dispensing table 24 of the transporter 20. The sample dispensing table 30 also has the function of dispensing the sample in the cuvette 153, which is held in the primary dispensing table 24 of the transporter 20, into the cuvette 154 held in the secondary dispensing table 23.

The two reagent dispensing arms 40 are provided to dispense the reagent in the reagent containers (not shown) held in the reagent tables 21 and 22 into the cuvette 154 of the secondary dispensing table 23.

The cuvette mover 50 is provided to move the cuvette 154 containing the analysis sample between the secondary dispensing table 23 of the transporter 20 and the cuvette loader 61 of the optical information obtainer 60.

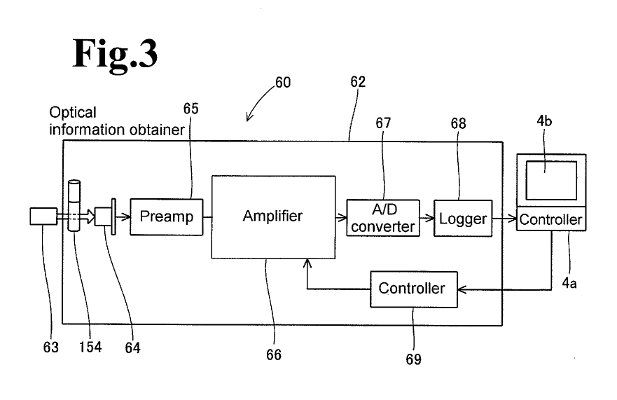

The optical information obtainer 60 has the functions of heating the analysis sample prepared by adding reagent to the sample, and optically measuring the analysis sample. The optical information obtainer 60 is configured by a cuvette loader 61, detection unit 62 (refer to FIG. 3) disposed below the cuvette loader 61, and a lamp unit 63 as a light source for directing light to the detection unit 62. The cuvette loader 62 is provided with a plurality of insertion holes 61a for inserting the cuvettes 154. The cuvette loader 61 has a built-in heating mechanism (not shown in the drawing) for heating a cuvette 154 loaded in the insertion holes 61a to a predetermined temperature.

As shown in FIG. 3, the detection unit 62 of the optical information obtainer 60 is capable of optically measuring the analysis sample in the cuvette 154 inserted in the insertion holes 61a (refer to FIG. 2) under a plurality of conditions. The detection unit 62 includes a photoelectric conversion element 64, preamp 65, amplifier 66, ND converter 67, logger 68, and controller 69.

The photoelectric conversion element 64 has the functions of receiving the light from the halogen lamp 631k of the lamp unit 63 (described later) transmitted through the analysis sample within the cuvette 154 inserted in the insertion hole 61a of the cuvette loader 61, detecting the received light, and converting the detected light to electrical signals. The preamp 65 is provided to amplify the electrical signal from the photoelectric conversion element 64. The amplifier 66 is provided to further amplify the electrical signal from the preamp 65. The amplifier 66 is capable of switching operations via control signals received from the controller 69.

The A/D converter 67 is provided to convert the electric signals (analog signals) from the amplifier part 66 to digital signals. The logger 68 has the function of temporarily storing the digital signal data from the ND converter 67. The logger 68 is electrically connected to the controller 4a of the control device 4, and has the function of transmitting the digital data obtained by the optical information obtainer 60 to the controller 4a of the control device 4.

As shown in FIG. 1, the lamp unit 63 is disposed below the cuvette supplier 10, and is accessible through an opening 1a provided in the side of the body of the detection device 2 of the sample analyzer 1. The opening 1a is opened and closed via the operation of a cover member 1b.

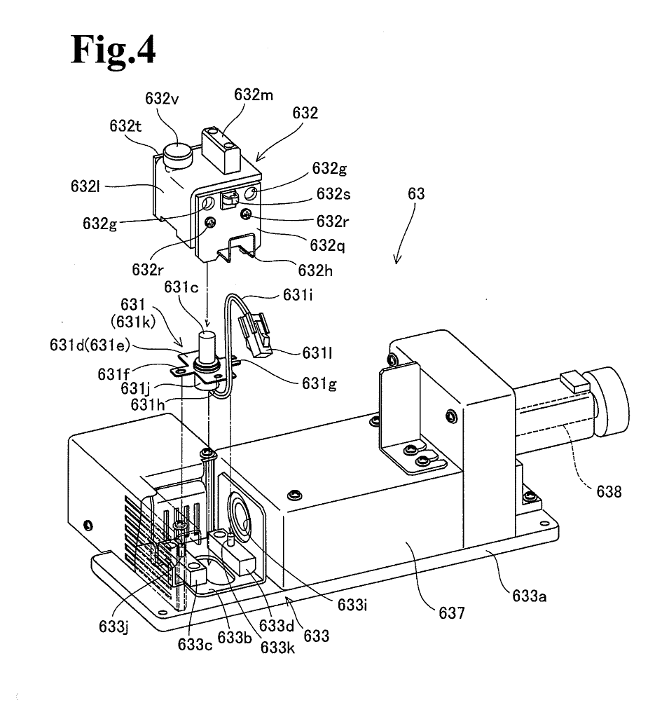

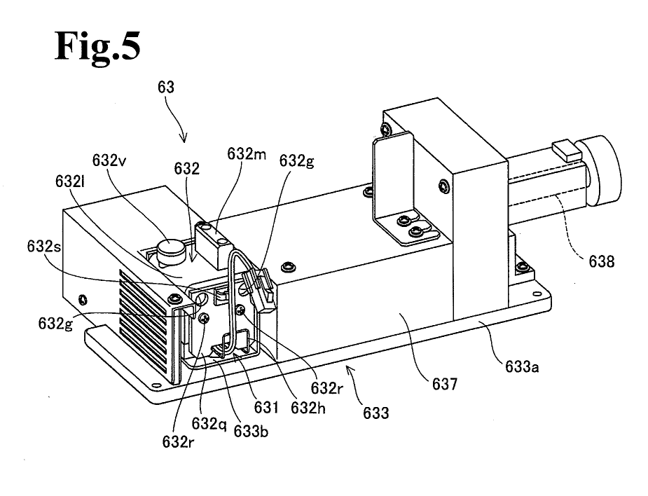

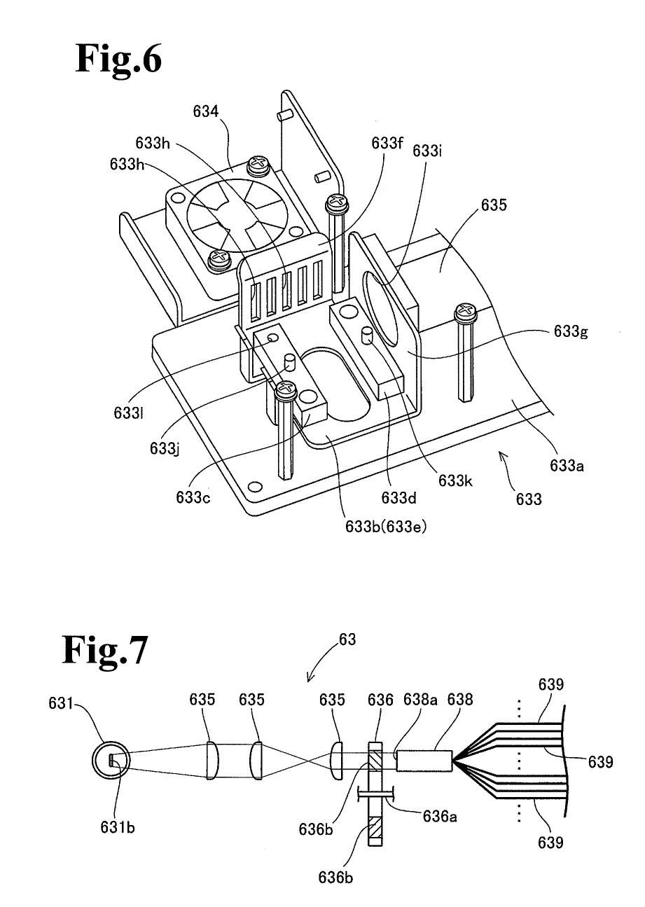

In the present embodiment, as shown in FIGS. 4 and 5, the lamp unit 63 has a lamp member 631, lamp housing 632 which accommodates the lamp member 631, base 633 for anchoring the lamp housing 632, fan 634 (refer to FIG. 6) for cooling the lamp housing 632, collecting lens 635 (refer to FIGS. 6 and 7), disk-shaped filter member 636 (refer to FIG. 7), casing 637 for accommodating the collecting lens 635 and filter member 636, optical fiber 638, and optical fiber splitter 639 (refer to FIG. 7). The collecting lens 635 is provided to direct the light irradiating from the halogen lamp 631k (described later) of the lamp member 631 to the optical fiber 638, as shown in FIG. 7. Note that the lamp member 631 is a disposable member. Although the lamp housing 632 is not disposable, the lamp housing 632 may also be discarded similar to the lamp member 631.

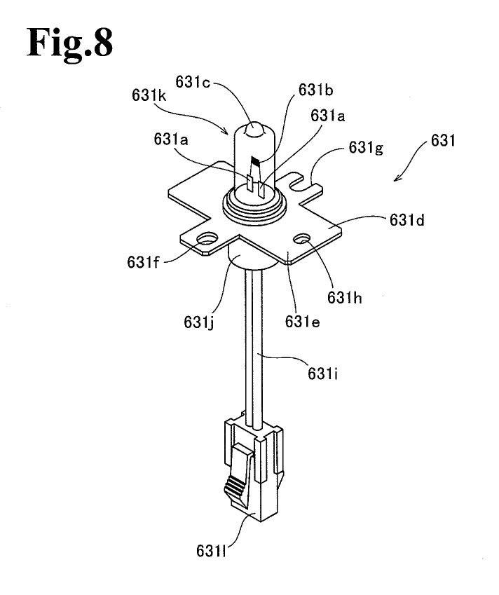

As shown in FIG. 8, the lamp member 631 includes a halogen lamp 631k, socket 631j, plate cap 631d provided on the socket 631j, wiring 631i, and connector 6311. The halogen lamp 631k includes an electrode 631a, filament 631b connected to the electrode 631a, silica glass bulb 631c housing the electrode 631a and filament 631b. The filament 631b of the halogen lamp 631k is configured of a material of mainly tungsten (W), and emits light when a current flows to the electrode 631a. The filament 631b has a flat light emitting surface. The interior of the bulb 631c of the halogen lamp 631k is filled with argon to which a small amount of halogen such as bromine or iodine has been added. Note that the halogen lamp 631k used in the present embodiment is a type with nonregulated positioning (lighting direction).

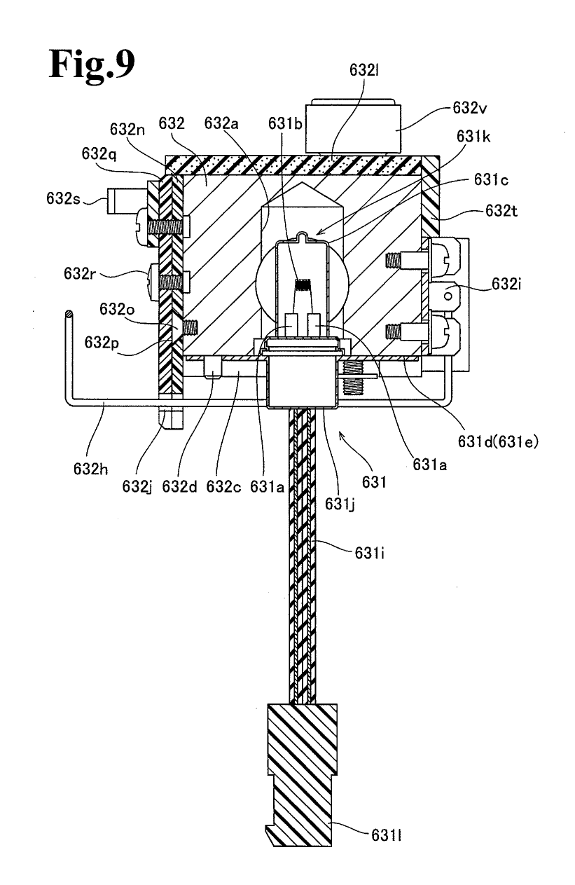

The plate cap 631d has a stainless steel plate 631e. The plate 631e is configured in a cross shape, and has a linkage hole 631f and notch 631g respectively capable of engaging a pair of knock pins 633j and 633k (refer to FIGS. 4 and 6) of the base 633 (described later). The plate 631e has an insertion hole 631h into which is inserted a protrusion 632d (refer to FIG. 9) provided on the lamp housing 632.

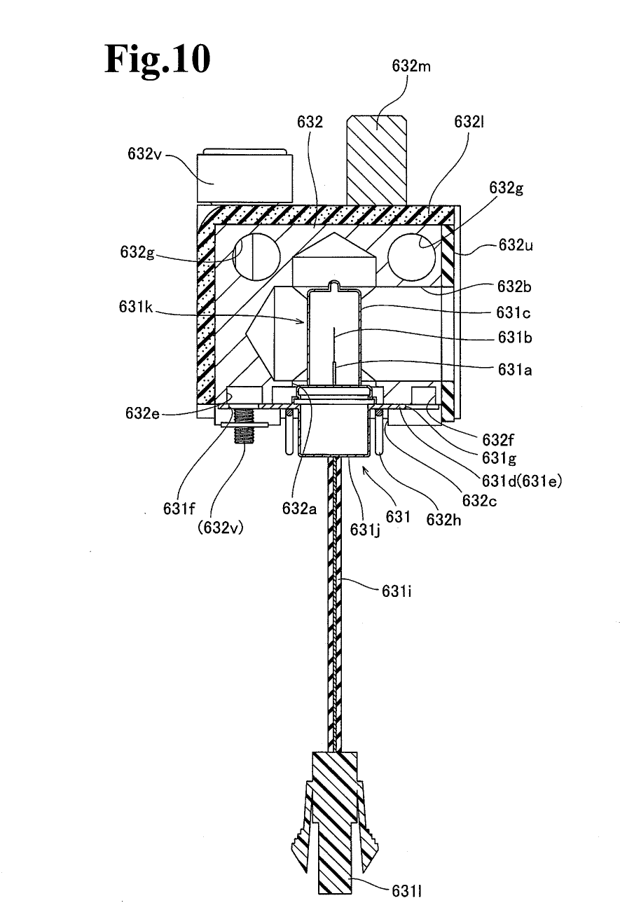

The lamp housing 632 is milled from an aluminum block, as shown in FIGS. 9 and 10. The lamp housing 632 has a housing hole 632a that houses the bulb 631c of the halogen lamp 631k when the bulb 631c is inserted from below, as shown in FIG. 9. The bulb 631c of the halogen lamp 631k is thus positioned above the wiring 631i of the lamp member 631. As shown in FIG. 10, the lamp housing 632 also has a guide hole 632b for guiding the light irradiated from the filament 631b of the halogen lamp 631k to the collecting lens 635 (refer to FIG. 7). The filament 631b of the halogen lamp 631k is deployed so that the flat light emitting surface faces the opening of the guide hole 632b, and so as to also face the light receiving surface 638a (refer to FIG. 7) of the optical fiber 638 (refer to FIG. 5).

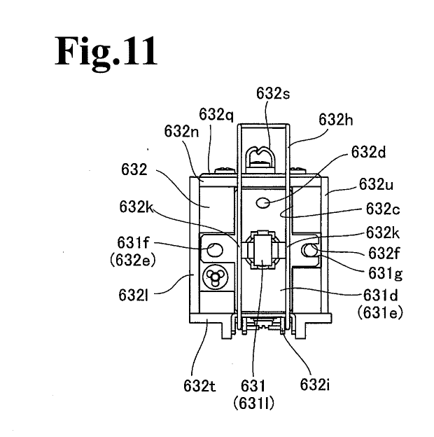

A cross-shaped channel 632c is provided in the vicinity of the housing hole 632a (refer to FIG. 10) of the lamp housing 632, as shown in FIGS. 10 and 11. The channel 632c is configured so as to allow the insertion of the flat cross-shaped plate 631e of the cap 631d of the lamp member 631. The channel 632c is also provided with a projection 632d, as shown in FIGS. 9 and 11. The projection 632d has the function of engaging the cap 631d inserted in the channel 632c at a predetermined position when the projection 632d is inserted (engages) in the insertion hole 631h of the plate 631e.

As shown in FIG. 10, a pair of pin holes 632e and 632f are formed in the channel 632c, as shown in FIGS. 10 and 11. The pair of pin holes 632e and 632f respectively correspond to the parts where the linkage hole 631f and notch 631g of the cap 631d are positioned when the plate 631e of the cap 631d is inserted into the channel 632c. Thus, the base 633, lamp member 631, and lamp housing 632 can be positioned together by inserting the knock pins 633j of the base 633 (described later) into the linkage hole 631f and pin hole 632e. The base 633, lamp member 631, and lamp housing 632 are also positioned together by inserting the knock pin 633k of the base 633 (described later) into the pin hole 632f and notch 631g of the cap 631d.

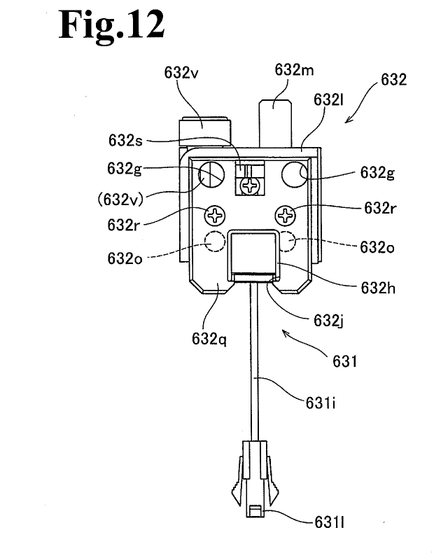

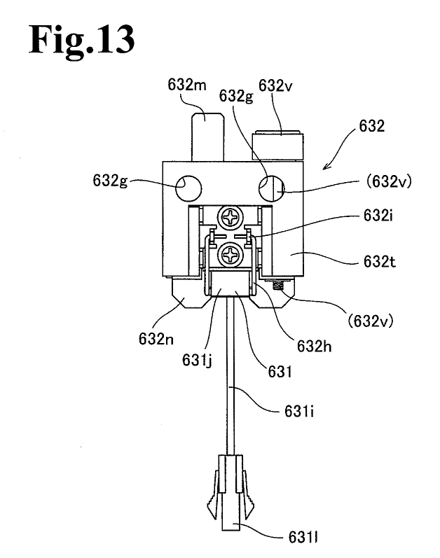

The lamp housing 632 also has two pass-through holes 632g that pass through the lamp housing 632 from the front to the back of the lamp housing 632, as shown in FIGS. 12 and 13. As shown in FIG. 5, the two pass-through holes 632g are formed so that the openings are directed in the direction of deployment of the fan 634 (refer to FIG. 6) when the lamp housing 632 is positioned on the base 633. As shown in FIG. 10, the pass-through holes 632g are connected to neither the housing hole 632a nor guide hole 632b so that the airflow of the fan 634 does not flow to either the housing hole 632a or guide hole 632b.

The aluminum lamp housing 632 is treated with an alumite process so as to be entirely black in color. Excessive heating of the halogen lamp 631k can therefore be prevented because radiant heating is suppressed when the halogen lamp 631k emits light.

The width of the channel 632c of the aluminum lamp housing 632 may be greater than the width of the plate 631e of the stainless steel cap 631d when the cap 631d of the lamp member 631 is engaged to the lamp housing 632 during assembly. The width of the channel 632c of the lamp housing 632 is configured to be approximately equal to the width of the plate 631e of the cap 631d engaged to the channel 632c via the difference of the thermal expansion coefficients between the aluminum lamp housing 632 and the stainless steel cap 631d when they are heated by the light emitted by the halogen lamp 631k when the halogen lamp 631k is in use. Thus, it is possible to regulate the position of the plate 631e of the cap 631d inserted in the channel 632c of the lamp housing 632.

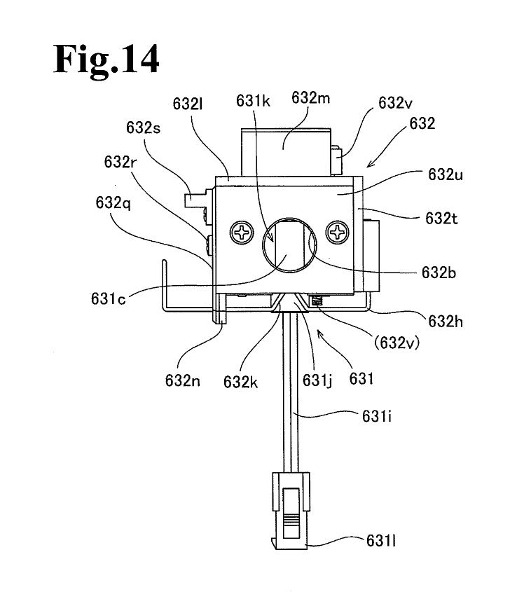

The lamp housing 632 is provided with a spring member 632h that mounts the lamp member 631 to the lamp housing 632, as shown in FIGS. 9 through 14. Specifically, a bracket 632i capable of supporting one end of the spring member 632h so as to be rotatable is provided on the lamp housing 632 as shown in FIGS. 9 and 13, and a hook 632j capable of engaging the other end of the spring member 632h is mounted as shown in FIGS. 9 and 12. As shown in FIG. 14, the spring member 632h exerts a force on the back surface (bottom surface) of the plate 631e (refer to FIG. 10) of the cap 631d toward the channel 632c of the lamp housing 632 (refer to FIG. 10) via a peak-shaped curved part 632k on the lamp housing 632 side when the other side is engaged with the hook 632j (refer to FIG. 9). Thus, the lamp member 631 can be mounted to the lamp housing 632 without use of tools.

A heat shield member 632l formed of sponge with heat resistant properties is mounted on the lamp housing 632, as shown in FIGS. 5 and 10. The heat shield member 632l is positioned so as to cover the surface on the top of the lamp housing 632 and the opposite side from the guide hole 632b. A handle 632m formed of polyacetal is provided on the top of the lamp housing 632 so as to be grippable by a user when removing the lamp housing 632 from the base 633 (refer to FIG. 4).

As shown in FIG. 9, a polycarbonate plate 632n is mounted on the surface directed toward the opening 1a of the body of the detection device 2 (refer to FIG. 1) of the sample analyzer 1 when the lamp housing 632 is mounted on the base 633. The plate 632n is provided with a screw hole 632p capable of accommodating part of the head of a machine screw 632o, and the plate 632n is mounted on the lamp housing 632 such that the machine screw 6320 is accommodated in the screw hole 632p. A polycarbonate plate member 632q is also mounted on the outside of the plate 632n. The plate member 632q is mounted on the plate member 632n via a screw member 632r. Thus, excessive heating is prevented at the part shielded by the plate member 632n, that is, the part directed toward the opening 1a of the lamp unit 63 since the plate member 632q is mounted on the lamp housing 632 through the plate member 632n. The screw member 632r does not make direct contact with the lamp housing 632 and is only screwed into the plate member 632n. Thus, the screw member 632r is prevented from becoming excessively hot even when the lamp housing 632 itself is very hot. Note that the hook 632j is integratedly formed with the plate members 632n and 632q.

A wire holder 632s is mounted on the plate member 632q to hold the wiring 631i when the lamp member 631 is mounted on the lamp housing 632, as shown in FIGS. 5 and 9.

A polycarbonate plate member 632t is mounted on the surface of the lamp housing 632 on which the bracket 632i is mounted to support the spring member 632h. As shown in FIGS. 10 and 14, a polycarbonate plate member 632u is mounted on the surface of the lamp housing 632 provided with the guide hole 632b.

The head part of the screw member 632v, which screws the lamp housing 632 into the mount member 633c of the base 633, is mounted on the top surface of the lamp housing 632, as shown in FIGS. 9 and 10. The threaded part of the screw member 632v is deployed in the lamp housing 632 so as to protrude below the lamp housing 632, and is provided to anchor the lamp housing 632 to the base 633. The head part of the screw member 632v is a knob which is rotated manually and directly by the user.

In the present embodiment, the base 633 is mounted to the body of the detection device 2 (refer to FIG. 1) of the sample analyzer 1 so that the plate member 633a (described later) is positioned on the bottom side, and is provided to standardize the placement positions of the lamp member 631, lamp housing 632, collecting lens 635 (refer to FIG. 7), filter member 636 (refer to FIG. 7), optical fiber 638 and the like, as shown in FIG. 4. The base 633 is mainly configured by the plate member 633a, metal bracket 633b mounted on the plate member 633a, and pair of mount members 633c and 633d.

The bracket 633b is configured by a bottom 633e mounted on the plate member 633a, and two walls 633f and 633g extending from the bottom 633e to the top side, as shown in FIG. 6. The bottom 633e is tightened both to the plate member 633a from the back side of the plate member 633a and the mount members 633c and 633d via a screw member (not shown). The wall 633f is provided on the side on which the fan is disposed, and has a plurality of slit-like ventilation holes 633h. The wall 633g is provided on the side on which the housing case 637 (refer to FIG. 4) is disposed, and has an opening 633i facing the guide hole 632b (refer to FIG. 14) of the lamp housing 632.

A knock pin 633j which has relatively high dimensional precision is mounted on the mount member 633c. The knock pin 633j is configured to engage the linkage hole 631f of the plate 631e of the cap 631d when the lamp housing 632 bearing the mounted lamp member 631 is anchored to the base 633. That is, the knock pin 633j has the function of positioning the lamp member 631 relative to the base 633. The knock pin 633j also is configured to be inserted into the pin hole 632e of the lamp housing 632 when the lamp housing 632 bearing the mounted lamp member 631 is anchored to the base 633. Thus, the lamp housing 632 can be positioned relative to the base 633. The mount member 633c has a screw hole 6331 threaded to accept the screw member 632v in order to securely anchor the lamp housing 632 to the base 633.

A knock pin 633k which has relatively high dimensional precision is mounted on the mount member 633d. The knock pin 633k is configured to engage the notch 631g of the plate 631e of the cap 631d when the lamp housing 632 bearing the mounted lamp member 631 is anchored to the base 633. That is, the knock pin 633k has the function of positioning the lamp member 631 relative to the base 633. The knock pin 633k also is configured to be inserted into the pin hole 632f of the lamp housing 632 when the lamp housing 632 bearing the mounted lamp member 631 is anchored to the base 633. Thus, the lamp housing 632 can be positioned relative to the base 633. The presence of a gap can be prevented between the wall 633g of the bracket 633b and the surface (plate member 632u) provided with the guide hole 632b on the lamp housing 632 when the lamp housing 632 is mounted while thus positioned relative to the base 633.

The lamp housing 632 bearing the deployed lamp member 631 thus can be accurately mounted such that the filament 631b of the halogen lamp 631k is positioned above the electrode 631a.

The filter member 636 is rotatable on a shaft 636a, as shown in FIG. 7. The filter member 636 is provided with a plurality of filters 636b which have different transmission wavelengths. Light of a plurality of different wavelengths can be sequentially supplied to the light receiving surface 638a of the optical fiber 638 because the light from the halogen lamp 631k can be sequentially transmitted through the plurality of filters 636b which have different transmission wavelengths by rotating the filter member 636 that has a plurality of filters 636b of different transmission wavelengths.

The optical fiber splitter 639 is provided to supply light to the cuvettes 154 inserted in the plurality of insertion holes 61a of the cuvette loader 61 by splitting the light from the optical fiber 638.

The cuvette discard unit 70 is provided to dispose of cuvettes 153 from the transporter 20. The cuvette discard unit 70 moves the cuvettes 153 and 154 of the transporter 20 to the discard box 72 via a discard catcher 71.

The fluid section 80 shown in FIG. 1 is provided to supply a liquid such as washing liquid and the like to nozzles provided on the sample dispensing arm 30 and two reagent dispensing arms 40 when the shutdown process is performed to shutdown the sample analyzer 1.

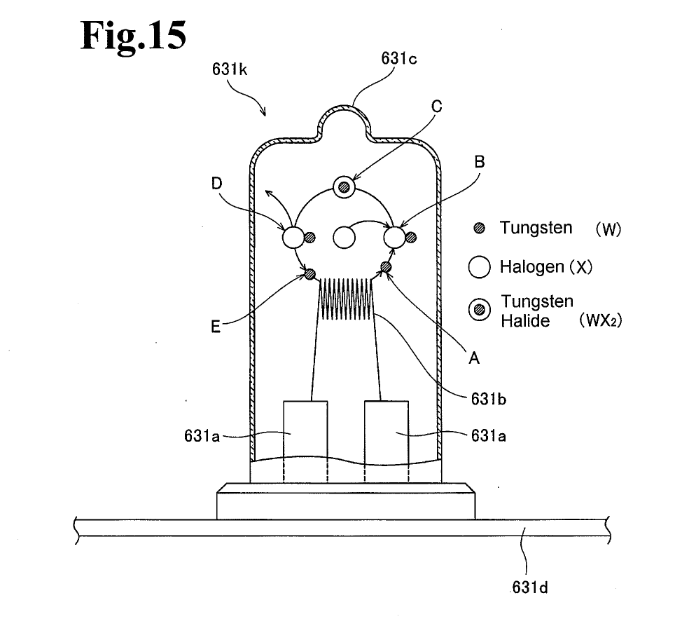

The halogen cycle occurring in the bulb 631c of the halogen lamp 631k when the halogen lamp 631k is in use (emitting light) is described below with reference to FIG. 15.

First, the filament 631b emits light and the filament 631b is heated to a high temperature by a current supplied to the filament 631b through the electrode 631a. Tungsten (W), a component of the filament 631b, is thus dissociated from the filament 631b as represented by A of FIG. 15.

The dissociated tungsten (W) reacts with the halogen (X) added to the argon in the bulb 631c as indicated by B in FIG. 15, so as to form tungsten halide (WX₂) as indicated by C in FIG. 15.

When the tungsten halide (WX₂) returns to the vicinity of the filament 631b, the tungsten (W) and halogen (X) are dissociated via the heat of the filament 631b. The dissociated halogen (X) returns to the bulb 631c, and the tungsten (W) is absorbed by the filament 631b.

The halogen lamp 631k is configured to return the tungsten (W) dissociated from the filament 631b back to the filament 631b via the halogen cycle.

Note that in the halogen cycle the dissociated tungsten (W) adhered to the inner surface of the bulb 631c when the temperature of the bulb 631c positioned in the space in which the halogen cycle occurs falls below a predetermined temperature. The tungsten (W) of the filament 631b becomes insufficient and the service life of the filament 631b is shortened when the tungsten (W) adheres to the inner surface of the bulb 631c due to the low temperature of the bulb 631c. On the other hand, when the temperature of the bulb 631c becomes excessively hot, the filament 631b is corroded by the halogen (X) due to the excessive activity of the halogen (X) in the bulb 631c. As a result, the service life of the filament 631b is shortened.

In the present embodiment, the temperature can be maintained in a predetermined temperature range by suitably configuring the lamp housing 632 and the surrounding parts to produce a smooth halogen cycle by the halogen lamp 631k.

In the present embodiment, the service life of the filament 631b (average service life) can be increased by deploying the halogen lamp 631k such that the filament 631b of the halogen lamp 631k is positioned above the electrode 631a. Described below are the principles involved in extending the service life (average service life) of the filament 631b by deploying the halogen lamp 631k such that the filament 631b is positioned above the electrode 631a.

There is a space above the filament 631b in the bulb 631c, and the argon that was added to the halogen is present in this space. When the filament 631b emits light, tungsten is dissociated from the filament 631b, and this dissociated tungsten spreads to the space above the filament 631b in the bulb 631c. When halogen lamp 631k emits light, the heat generated by the filament 631b moves upward and spreads through the entirety of the inner surface of the bulb 631c above the filament 631b opposite the electrode 631a. As a result, a drop in temperature is prevented at the top part of the bulb 631c in the space in which the halogen cycle occurs.

Therefore, the halogen (X) is prevented from adhering to the inner surface of the bulb 631c, and the halogen cycle occurs sufficiently at the top part of the bulb 631c. As a result, the service life (average service life) of the filament 631b can be increased because this arrangement prevents the insufficiency in which the tungsten is not returned to the filament 631b and the halogen cycle collapses.

The result of service life tests (comparative tests) of the halogen lamp 631k of the present embodiment are described below, both when the filament 631b of the halogen lamp 631k is positioned above the electrode 631a and, for comparison, when the filament 731b of the halogen lamp 731 is positioned below the electrode 731a referring to FIGS. 15 and 16. The present inventors have verified that the average service life of the halogen lamp can be extended when the filament 631b of the halogen lamp 631k is disposed above the electrode 631a. Details are described below.

The horizontal axis of the graph in FIG. 16 represents the continuous lighted time of the halogen lamps of the present embodiment and the comparative example. The vertical axis of the graph in FIG. 16 represents the percentage value (number) corresponding to the total number of pulses during the experiment. The curve expressed by the solid line in the graph of FIG. 16 represents the Gaussian distribution calculated based on the actual measured service life of a plurality of continuously lighted samples (halogen lamp 631k) with the filament 631b of the halogen lamp 631k positioned above the electrode 631a as in the present embodiment. The curve expressed by the dashed line in the graph of FIG. 16 represents the Gaussian distribution calculated based on the actual measured service life of a plurality of continuously lighted samples (halogen lamp 731) with the filament 731b of the halogen lamp 731 positioned below the electrode 731a as in the comparative example.

It has been confirmed via the two Gaussian distributions that the improvement of positioning the filament 631b of the halogen lamp 631k above the electrode 631a as in the present embodiment increased the service life of the halogen lamp 631k compared to the halogen lamp 731 of the comparative example. That is, heat generated by the filament 731b spread (upward) above the electrode 731a due to the upward movement of the heat when the filament 731a of the halogen lamp 731 was positioned below the electrode 731a in the comparative example. As a result, the temperature was reduced in the part on the opposite side from the electrode 731a of the bulb 731c (space in which the halogen cycle occurs) due to insufficient heat generated from the filament 731b in the part (part below the bulb 731c in the comparative example) on the opposite side from the electrode 731a of the bulb 731c. Adhesion of tungsten (W) to the inner surface of the bulb 731c of the comparative example was visually confirmed. Conversely, the positioning of the halogen lamp 631k of the present embodiment produced a smooth halogen cycle.

In the present embodiment described above, a reduction of the temperature is prevented in the part above the filament 631b of the glass bulb 631c covering the filament 631b and electrode 631a because the heat generated by filament 631b is spread above the filament 631b when the halogen lamp 631k is in use by installing the lamp housing 632 in which the halogen lamp 631k is disposed such that the filament 631b of the halogen lamp 631k is positioned above the electrode 631a. Thus, when the halogen lamp 631k is in use, the tungsten dissociated from the filament 631b that contains tungsten combines with the halogen within the bulb in the space above the filament 631b within the bulb 631c to form halide, and thereafter the halogen and tungsten dissociate again in the vicinity of the filament 631b and the dissociated tungsten is returned to the filament 631b in the so-called halogen cycle; this arrangement prevents the insufficiency in which the tungsten fails to return to the filament 631b causing the halogen cycle to collapse due to the adhesion of the tungsten to the inner surface of the glass bulb 631c due to the crop in temperature in the part of the bulb 631c above the filament 631b. The service life (average service life) of the filament 631b can thus be increased since reduction of the filament 631b is suppressed. As a result, the frequency of replacement of the lamp member 631 is reduced.

In the present embodiment described above, positional dislocation of the lamp member 631 relative to both the base 633 and the lamp housing 632 is prevented by maintaining the lamp member 631 in a position relative to both the base 633 and lamp housing 632 by maintaining the lamp member 631 interposed between the base 633 and lamp housing 632 when the lamp housing 632 is anchored to the base 633.

In the present embodiment described above, the light reliably impinges the light receiving surface 638a of the optical fiber 638 via the broad irradiation range produced by the flat light emitting surface of the filament 631b even when the mounting position of the lamp member 631 is slightly shifted because the flat light emitting surface of the filament 631b is positioned facing the light receiving surface 638a of the optical fiber 638. The light receiving surface 638a of the optical fiber 638 is uniformly irradiated, and equal amounts of light are split via the optical fiber splitter 639.

In the present embodiment described above, shifting of the cap 631d of the lamp member 631 is prevented in the rotational direction relative to the channel 632c because the cap 631d is regulated relative to the rotation direction by the insertion of the cross-shaped plate 631e into the channel 632c by providing the cross-shaped plate 631e and providing the cross-shaped channel 632c capable of accepting the insertion of the plate 631e of the cap 631 in t5hge lamp housing 632. Thus, the flat light emitting surface is prevented from shifting in the rotation direction even when using a filament 631b that has a flat light emitting part as the filament of the halogen lamp 631k.

In the present embodiment described above, there is minimal assembly error of the lamp member 631 and other member such as the optical fiber 638 because the lamp member 631 mounting on the base 633 is standardized by the knock pins 633j and 633k of the base 633 similar to the optical fiber 638 and other members positioned relative to the base 633 by providing the knock pins 633j and 633k for positioning the cap 631d of the lamp member 631 engaged to the lamp housing 632 when the lamp housing 632 is anchored to the base 633.

In the present embodiment described above, the structure of devices related to the optical system are simplified when the filament 631b is positioned such that the flat light emitting surface of the filament 631b faces the light receiving surface of the optical fiber compared to when the filament 631b with a flat light emitting surface is disposed in a horizontal direction (direction horizontal to the filament 631b and electrode 631a) because the filament 631b is arranged in the vertical direction (direction perpendicular to the direction of the filament 631b and electrode 631a). That is, the filament 631b tends to curl due to its own weight making it difficult for a stable amount of light to irradiate the light receiving surface of the optical fiber when the flat light emitting surface of the filament 631b is arranged horizontally such that the flat light emitting surface faces the light receiving surface of the optical fiber. When the flat light emitting surface of the filament 631b is arranged in a horizontal direction facing upward or downward, the structure of devices related to the optical system are complicated because a mirror or other device is required to bend the light from the might emitting surface of the filament 631b in the direction of the light receiving surface of the optical fiber. In the present embodiment, the structure of devices related to the optical system are simplified and a stable amount of light irradiates the light receiving surface of the optical fiber because mirrors and the like are not required to bend the light emitted from the filament 631b since the filament 631b is disposed in a vertical direction.

In the present embodiment described above, direct contact of the user and the bulb 631c of the halogen lamp 631k is prevented even when performing a replacement operation immediately after the halogen lamp 631k has stopped emitting light because the lamp member 631 can be replaced when the halogen lamp 631k has deteriorated. Therefore, the burn injuries are prevented from the heat generated by the halogen lamp 631k.

The above embodiment is offered as an example and should not to be considered limiting in any way. The scope of the present invention is defined by the scope of the claims and not be the description of the embodiment, and includes all modifications within the scope of the claims and the meanings and equivalences therein.

For example, although the present embodiment has been described by way of example in which the lamp housing is anchored to the base, the present invention is not limited to this arrangement inasmuch as the lamp housing may also be directly mounted on the detection device 2.

Although the present embodiment has been described by way of example in which the halogen lamp is mounted to the lamp housing via a spring member, the present invention is not limited to this arrangement since, for example, the halogen lamp also may be mounted to the lamp housing by a member such as a screw member or the like rather than a spring member. The halogen lamp and lamp housing may also be integrated in a single unit.

Although the present embodiment has been described by way of example using a halogen lamp provided with a filament with a flat light emitting surface, the present invention is not limited to this arrangement since, for example, a halogen lamp provided with a spiral shaped filament, or other shaped filament may be used rather than a filament with a flat light emitting surface.

Although the present embodiment has been described by way of example in which two knock pins are provided to position the cap of the halogen lamp relative to the base, the present invention is not limited to this arrangement inasmuch as one or three or more knock pins may be used to position the cap of the halogen lamp relative to the base, or, for example, the cap of the halogen lamp may be positioned relative to the base by providing a channel in the base and engaging the cap in the channel rather than using knock pins.

Although the present embodiment has been described by way of example in which two pass-through holes are provided to cool the lamp housing, the present invention is not limited to this arrangement inasmuch as one or three or more pass-through holes also may be provided.

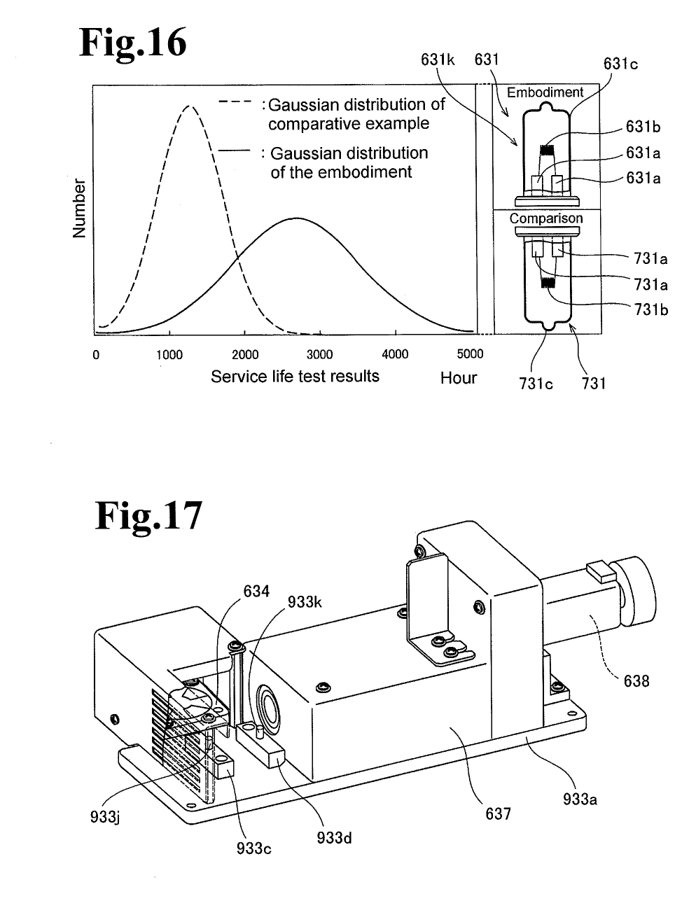

Although the present embodiment has been described by way of example in which the mount members 633c and 633d of the base 633 are mounted to the plate member 633a by tightening to both the bracket 633b and plate member 633a, the present invention is not limited to this arrangement since, for example, the mount members 633c and 633d also may be directly mounted to the plate member 933a as shown in the modification of FIG. 17.

The modification of the embodiment of the present invention shown in FIG. 17 provides mount members 933c and 933d for positioning the lamp member 631 (refer to FIG. 8). The mount members 933c and 933d respective mount directly on the plate member 933a of the base 933. The mount members 933c and 933d are adjusted such that the height position of the halogen lamp 631k matches the height position of the light receiving surface of the optical fiber 638 when the mount members 933c and 933d are disposed so that the lamp housing accommodating the lamp member 631 (refer to FIG. 8) is positioned by the knock pins 933j and 933k.

What is claimed is:

1. A sample analyzer, comprising:
a lamp member comprising a halogen lamp including an electrode and a filament connected to the electrode;
a holding mechanism for holding the lamp member such that the filament of the halogen lamp is positioned above the electrode;
a light receiver for receiving light irradiated from the halogen lamp through an analysis sample; and
an analysis unit for analyzing a component contained in the analysis sample based on the light received by the light receiver.

2. The sample analyzer of claim 1, wherein
the lamp member comprises:
a halogen lamp holder for holding the halogen lamp; and
a connector member for supplying electricity to the electrode of the halogen lamp held by the halogen lamp holder.

3. The sample analyzer of claim 1, wherein
the holding mechanism comprises
a lamp housing for covering the halogen lamp of the lamp member and to which the lamp member is detachably mounted, and
a base unit for detachably fixing the lamp housing to which the lamp member is being mounted; and
wherein the lamp housing is provided with a lamp insertion hole for inserting the halogen lamp from below the lamp housing, and the lamp housing comprises a surface including a light conductor for conducting the light irradiated from the halogen lamp to outside of the lamp housing.

4. The sample analyzer of claim 3, wherein
the lamp member is held between the base unit and the lamp housing when the lamp housing is fixed on the base unit.

5. The sample analyzer of claim 3, wherein
the lamp member comprises a first engaging part;
the lamp housing comprises a second engaging part capable of engaging with the first engaging part of the lamp member; and
the first engaging part engages with the second engaging part when the halogen lamp of the lamp member is inserted in the lamp insertion hole.

6. The sample analyzer of claim 5, wherein
the first engaging part of the lamp member comprises a flat part which has cross shape; and
the second engaging part of the lamp housing comprises a channel which has cross shape and is capable of engaging with the flat part of the first engaging part.

7. The sample analyzer of claim 5, wherein
the lamp housing comprises a fixing member for fixing the lamp member to the lamp housing when the first engaging part of the lamp member is engaged with the second engaging part of the lamp housing.

8. The sample analyzer of claim 5, wherein
the base unit comprises a positioner for positioning the first engaging part of the lamp member relative to the base unit.

9. The sample analyzer of claim 5, wherein
the first engaging part of the lamp member is formed with a material that has a larger thermal expansion coefficient than the second engaging part of the lamp housing; and
the first engaging part is smaller than the second engaging part.

10. The sample analyzer of claim 3, wherein
the lamp housing has a pass-through hole which is passing through the lamp housing from one side surface thereof to the opposite side surface thereof.

11. The sample analyzer of claim 10, wherein
the holding mechanism comprises an air blower for blowing air into the pass-through hole.

12. The sample analyzer of claim 3, wherein
the lamp housing comprises an insulating member covering at least part of an exterior surface of the lamp housing.

13. The sample analyzer of claim 3, wherein
the lamp housing comprises a handle on a top of the lamp housing, which a user can hold when detaching the lamp housing from the base unit.

14. The sample analyzer of claim 1, further comprising
a light transmitter comprising a light receiving surface for receiving the light irradiated from the halogen lamp, and transmitting the light received by the light receiving surface to the light receiver, wherein
the filament of the halogen lamp comprises a light emitting part which has flat shape; and
the light emitting part of the filament is arranged so as to face the light receiving surface of the light transmitter.

15. The sample analyzer of claim 1, wherein
the analysis sample is prepared from a blood sample and a reagent for blood coagulation analysis; and
the analysis unit analyzes the component related to coagulation function of the blood sample based on the light received by the light receiver.

16. A blood coagulation analyzer, comprising:
a sample preparing unit for preparing an analysis sample from a blood sample and a reagent for blood coagulation analysis;
a light source for irradiating light on the analysis sample prepared by the sample preparing unit;
a light receiver for receiving light irradiated from the light source through the analysis sample;
an analysis unit for analyzing a component contained in the analysis sample based on the light received by the light receiver, the component being related to coagulation function of the blood sample, wherein
the light source comprises:
a lamp member comprising a halogen lamp including an electrode and a filament connected to the electrode; and
a holding mechanism for holding the lamp member such that the filament of the halogen lamp is positioned above the electrode.

17. The blood coagulation analyzer of claim 16, wherein
the lamp member comprises:
a halogen lamp holder for holding the halogen lamp; and
a connector member for supplying electricity to the electrode of the halogen lamp held by the halogen lamp holder.

18. The blood coagulation analyzer of claim 16, wherein
the holding mechanism comprises
a lamp housing for covering the halogen lamp of the lamp member and to which the lamp member is detachably mounted, and
a base unit for detachably fixing the lamp housing to which the lamp member is being mounted; and
wherein the lamp housing is provided with a lamp insertion hole for inserting the halogen lamp from below the lamp housing, and the lamp housing comprises a surface including a light conductor for conducting the light irradiated from the halogen lamp to outside of the lamp housing.

19. The blood coagulation analyzer of claim 18, wherein
the lamp member is held between the base unit and the lamp housing when the lamp housing is fixed on the base unit.

20. The blood coagulation analyzer of claim 18, wherein
the lamp member comprises a first engaging part;
the lamp housing comprises a second engaging part capable of engaging with the first engaging part of the lamp member; and
the first engaging part engages with the second engaging part when the halogen lamp of the lamp member is inserted in the lamp insertion hole.
